(19) United States
(12) Patent Application Publication
(10) Pub. No.: US 2015/0039029 A1
(43) Pub. Date: Feb. 5, 2015

(54) IMPLANT DEVICE AND SYSTEM FOR STABILIZED FIXATION OF BONE AND SOFT TISSUE

(71) Applicant: Dallen Medical, Inc., San Clemente, CA (US)

(72) Inventor: Russell W. Wade, Laguna Niguel, CA (US)

(21) Appl. No.: 14/449,878

(22) Filed: Aug. 1, 2014

Related U.S. Application Data
(60) Provisional application No. 61/861,305, filed on Aug. 1, 2013.

(51) Int. Cl. A61B 17/04

(52) U.S. Cl. CPC A61B 17/0401; A61B 2017/0404
USPC 606/232

(57) ABSTRACT

A system for syndesmosis repair includes a flat band secured to bone by pulling a metal button through both the tibial and fibula bones, which button is then toggled into position to create tension, along the band, across the two bone segments to be fixated. Once the button is secure against the bone, flat suture tails against the lock at the initial insertion site serves to tension the band and bone segments in place. The system band offers syndesmosis repair with a knotless closure. The system is designed to provide this stabilized fixation for bone fractures, osteotomies, and arthrodesis, plus soft tissue to bone attachment, if desired. It is designed to apply a restorative fixation force across the tissue segments to stabilize them. The band's rigidity and compliant nature provides consistent and rigid fixation during the healing phase.

This application claims the benefit under 35 U.S.C. 119(e) of the filing date of Provisional U.S. Application Ser. No. 61/861,305, entitled Implant Device for Stabilized Fixation of Bone and Soft Tissue, filed on Aug. 1, 2013, and expressly incorporated herein by reference, in its entirety.

### BACKGROUND OF THE INVENTION

A syndesmosis is a slightly movable articulation where the contiguous bony surfaces are united by an interosseous ligament. If the syndesmosis is separated because of bone fracture, surgeons will sometimes fix the relevant bones together with a syndesmotic screw. The screw inhibits normal movement of the bones and, thereby, the corresponding joint or joints. When the natural articulation is healed, the screw may be removed. The tightrope fixation with elastic fiberwire suture on the other hand allows physiologic motion of the ankle and may be permanent.

Syndesmosis screws have significant problems, including loosening, breakage, the need for removal, and late diastasis. The present invention has been developed to address these problems, by providing a low profile implant device intended to facilitate stabilized fixation of tissues, including bone and soft tissue to bone, for syndesmosis repair, as well as other applications.

### SUMMARY OF THE INVENTION

The present inventive system, in one particular application, is designed for delivery through a pre-drilled hole through the tibia and fibula, for example, by means of a pilot needle delivery, exiting the skin opposite the initial insertion site. The system includes a flat band secured to bone by pulling a metal button through both the tibial and fibula bones, which button is then toggled into position to create tension, along the band, across the two bone segments to be fixated. Once the button is secure against the bone, suture attached to the needle can be cut and removed from the operative site. Pulling flat suture tails against the lock at the initial insertion site serves to tension the band and bone segments in place. The system band offers syndesmosis repair with a knotless closure. The system is designed to provide this stabilized fixation for bone fractures, osteotomies, and arthrodesis, plus soft tissue to bone attachment, if desired. It is designed, as discussed above, to apply a restorative fixation force across the tissue segments to stabilize them. The band's rigidity and compliant nature provides consistent and rigid fixation during the healing phase.

The present system is an assembly of four main components; a suture band, a narrow button, a lock, and a straight needle. The woven suture is made, in certain embodiments, from the same polyester (PET) as most sutures. The lock and narrow button may be manufactured, in certain embodiments, from 316 LVM stainless steel. The straight needle assembly is comprised of a PET suture tether and a guide cap. The system is designed to be a one-size-fits-all sterile implant.

The inventive system is intended for use as an adjunct in fracture repair involving metaphyseal and periarticular small bone fragments where screws are not indicated, and as an adjunct in external and intramedullary fixation systems involving plates and rods, with fracture braces and casting. Specifically, the inventive band is intended to provide fixation during the healing process following a syndesmotic trauma, such as fixation of syndesmosis (syndesmosis disruptions) in connection with Weber B and C ankle fractures. However, the system may be used in treating syndesmosis disruptions with or without associated ankle fractures. Although initially developed to address ankle syndesmosis injuries and fibula and tibia fixation, additional applications may include the shoulder (CC ligament repair) and ACL/PCL repair, for example. Use of the band of the present system provides greater bone compliance and does not restrict movement, such as may occur with screw fixation.

More particularly, there is provided, in one aspect of the invention, an implant system for stabilized fixation of tissue, which comprises a button having a slot, a band threaded through the slot, and a lock comprising a frame having an intake opening, a movable member, and an exit slot. A free end of material forming the band enters the lock through the intake opening, extends around the movable member, and then through the exit slot. The movable member is movable between an open position and a locking position. The button comprises a pair of slots and the band is threaded through each of the pair of slots in sequence, so that the band is wrapped around a portion of the button separating the two slots.

The band may be formed of suture material, more particularly comprising a length of flat suture material threaded through the pair of slots of the button and through the intake opening and slots of the lock so that between the button and the lock the band comprises the length of suture doubled over itself.

The lock further comprises a plate on which the movable member is disposed, the plate having a first configuration when the movable member is in its open position and a second configuration when the movable member is in its locking position, the plate being movable from its first configuration to its second configuration responsive to applied tension exceeding a predetermined value. The movable member, which comprises a bar in illustrated embodiments, is adapted to move toward the exit slot when it is moved from its open position to its locking position to narrow an effective width of the slot and to thereby clamp the free end of the suture band extending through the slot between the movable member and an inside surface of the outer portion of the frame to lock the suture band free end in place.

Advantageously, the lock is designed to clamp the free end of the suture band in place at multiple spaced lock points. The first lock point is within the exit slot at a point where the movable member and the inside surface of the outer portion of the frame first clamp the suture band in place, and a second lock point, spaced distally from the first lock point, is created by a corner formed on the inside surface of the outer portion of the frame, so that the suture band is engaged between the corner and the movable member. A third lock point, spaced distally from the second lock point, is created by engagement of the suture band with a row of teeth, comprises a suture comb, disposed on a surface of the lock frame.

In disclosed embodiments, the frame comprises a second movable member and a second exit slot, the two exit slots being disposed on opposite sides of the intake opening adjacent to respective opposed outer portions of the frame. The construction of these two exit slots and associated movable members is substantially similar, in order to simultaneously clamp each of two free ends of the suture band extending through the lock.

Additionally, the lock comprises a second suture comb, each suture comb being disposed on the frame of the lock adjacent to respective exit points for the free ends of the suture bands from each of the two exit slots.

As noted above, the implant system further comprises a suture comb disposed on a surface of the frame. The suture comb comprises a row of teeth, and preferably multiple rows of teeth, for engaging suture passing thereover, and clamping the suture in place.

In another aspect of the invention, there is provided a lock for use in an implant system for stabilized fixation of tissue. The lock comprises a frame having an intake opening, a movable member, and an exit slot, for creating a path for a band of material to extend along, the path extending from the intake opening, around the movable member, and exiting through the exit slot, the exit slot being defined in part by an inner surface of the frame. The movable member is movable between an open position and a locking position.

The lock further comprises a plate on which the movable member is disposed, the plate having a first configuration when the movable member is in its open position and a second configuration when the movable member is in its locking position, the plate being movable from its first configuration to its second configuration responsive to applied tension exceeding a predetermined value. The movable member is adapted to move toward the exit slot when it is moved from its open position to its locking position to narrow an effective width of the slot and to thereby clamp the free end of a suture band extending through that slot between the movable member and an inside surface of the outer portion of the frame to lock the suture band free end in place.

Advantageously, the lock is designed to clamp the free end of the suture band in place at multiple spaced lock points. The first lock point is within the exit slot at a point where the movable member and the inside surface of the outer portion of the frame are first adapted to clamp the suture band in place, and a second lock point, spaced distally from the first lock point, is created by a corner formed on the inside surface of the outer portion of the frame, so that the suture band is adapted to be engaged between the corner and the movable member. A third lock point, spaced distally from the second lock point, and created by engagement of a suture band, extending along the pathway through the lock, with a row of teeth, comprises a suture comb, disposed on a surface of the lock frame.

In disclosed embodiments, the frame comprises a second movable member and a second exit slot, the two exit slots being disposed on opposite sides of the intake opening adjacent to respective opposed outer portions of the frame. The construction of these two exit slots and associated movable members is substantially similar, in order to simultaneously clamp each of two free ends of the suture band extending through the lock.

The lock comprises a second suture comb, each suture comb being disposed on the frame of the lock adjacent to respective exit points for the free ends of the suture bands from each of the two exit slots.

As noted above, the implant system further comprises a suture comb disposed on a surface of the frame. The suture comb comprises a row of teeth, and preferably multiple rows of such teeth, for engaging suture passing thereover, and clamping the suture in place.

In still another aspect of the invention, there is provided a lock for use in an implant system for stabilized fixation of tissue. The lock comprises a frame having an opening for receiving a length of material therethrough, a movable member about which the length of material may extend, and a slot through which the length of material may exit after passing about the movable member. The slot is defined in part by an inner surface of the frame. The movable member is movable between an open position and a locking position, the open position being adapted to permit free movement of the length of material through the lock and the locking position being adapted to clamp the length of material in place. The lock is constructed and designed to clamp the length of material at a plurality of locking points, spaced from one another, when the movable member is in said locking position.

In yet another aspect of the invention, there is disclosed a method for effecting repair of tissue using a tissue fixation band apparatus comprising a lock, a button, and a suture band, under tension, extending between the lock and the button. The method comprises steps of pulling the button through a hole extending through the tissue to be repaired, until it exits a distal surface of the tissue and engages the distal surface of the tissue, pulling the suture band on each end to locate the lock in position on a proximal surface of the tissue, applying tension to free ends of the suture band exiting from the lock of the fixation band apparatus to tension the suture band to a desired level, and causing a movable clamping member within the lock to move to a locking position upon application of the desired tension level to the suture band, to thereby engage the lock and clamp the suture band in place at the desired tension level.

Advantageously, the causing step creates a plurality of spaced locking points along a length of the suture band free end extending through the lock.

The invention, together with additional features and advantages thereof, may best be understood by reference to the following description taken in conjunction with the accompanying illustrative drawings.

### BRIEF DESCRIPTION OF THE DRAWINGS

FIG. 1 is an isometric view of a tissue fixation band apparatus constructed in accordance with the principles of the present invention;

FIG. 2 is a top view of the apparatus of FIG. 1, with its lock in an open position;

FIG. 3 is a cross-sectional view taken along lines 3-3 of FIG. 2;

FIG. 4 is an isometric view of the apparatus of FIGS. 2 and 3, with the lock in its open position;

FIG. 5 is a top view similar to FIG. 2, showing the apparatus of FIG. 1 with its lock in a closed and locked position;

FIG. 6 is a cross-sectional view taken along lines 6-6 of FIG. 5;

FIG. 7 is an isometric view of the apparatus of FIGS. 5 and 6, with the lock in its closed and locked position;

FIG. 8 is an isometric view of one embodiment of the lock of the present invention, shown in its closed and locked position;

FIG. 9 is an isometric view similar to FIG. 8, showing the lock in its open position;

FIG. 10 is an isometric view similar to FIG. 8, showing the lock in its closed and locked position, from a slightly different perspective so that plates of the lock are more clearly visible;

FIG. 11 is an isometric view of the lock shown in FIG. 10, from the side thereof;

FIG. 12 is a top view of the lock of FIGS. 8-11;

FIG. 13 is a top view of the narrow button of the invention;

FIG. 14 is an isometric view of the narrow button shown in FIG. 13;

FIG. 15 is an end view of the narrow button of FIGS. 13-14;

FIG. 16 is an isometric schematic view illustrating the inventive apparatus in combination with a needle guide and suture loop for positioning the apparatus in place during a procedure; and

FIG. 17 is an isometric schematic view similar to FIG. 18, showing the apparatus, including needle guide and suture loop, in place at an operative site during a Syndesmosis procedure.

### DESCRIPTION OF THE PREFERRED EMBODIMENT

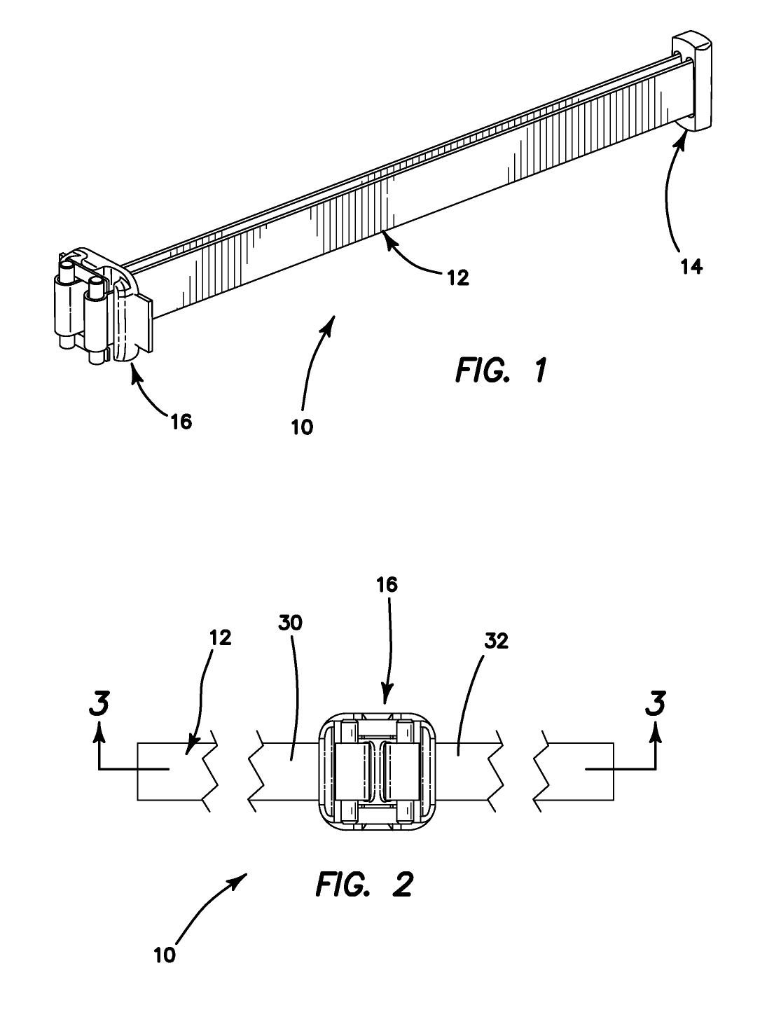

Referring more particularly to the drawings, there is shown in FIG. 1 a tissue fixation band apparatus 10 constructed in accordance with the principles of the present invention. The apparatus 10 comprises a band 12, a narrow button 14, and a lock 16.

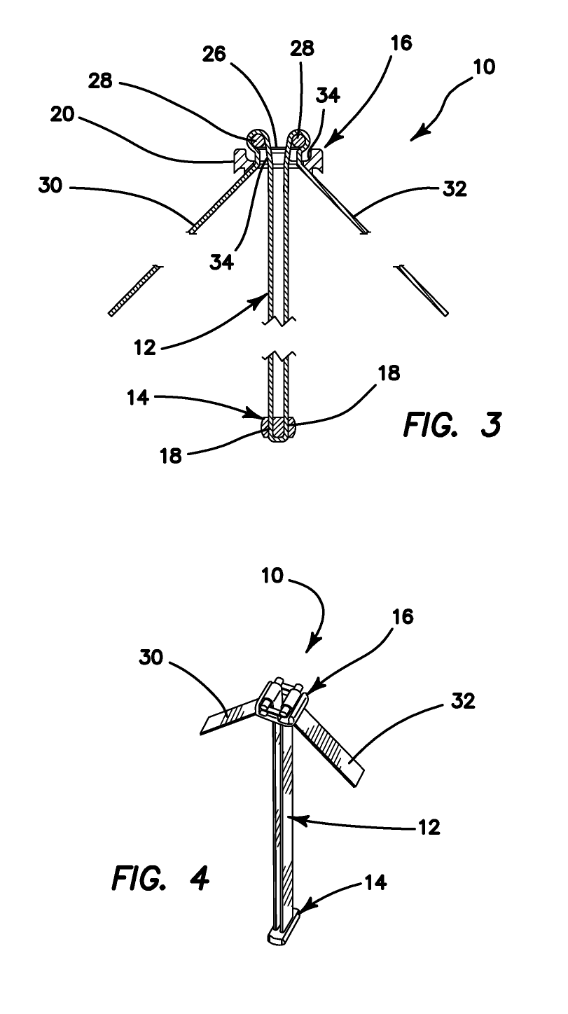

FIGS. 2-4 illustrate the apparatus 10 of FIG. 1 through different views of the apparatus 10 with the lock 16 in an open orientation. As illustrated, FIG. 3 is a cross-sectional view taken along lines 3-3 of FIG. 2. As shown in FIG. 3, there are slots 18 extending through the narrow button 14, through which the band 12, comprising suture which in certain embodiments is woven polyester made from PET (PolyEthylene Terephthalate), is threaded. In one embodiment for certain applications, the band 12 is thirty-six (36) inches in length, though both the length and material of the suture band 12 may be varied in accordance with performance preferences and applications. The lock 16 and the narrow button 14 may be made of 316 LVM stainless steel, or other suitable biocompatible material.

In the illustrated embodiment, the band 12 has an approximate three (3) inch length between the narrow button 14 and the lock 16, which is nominally set and adjustable by the physician per the anatomical requirements of the patient.

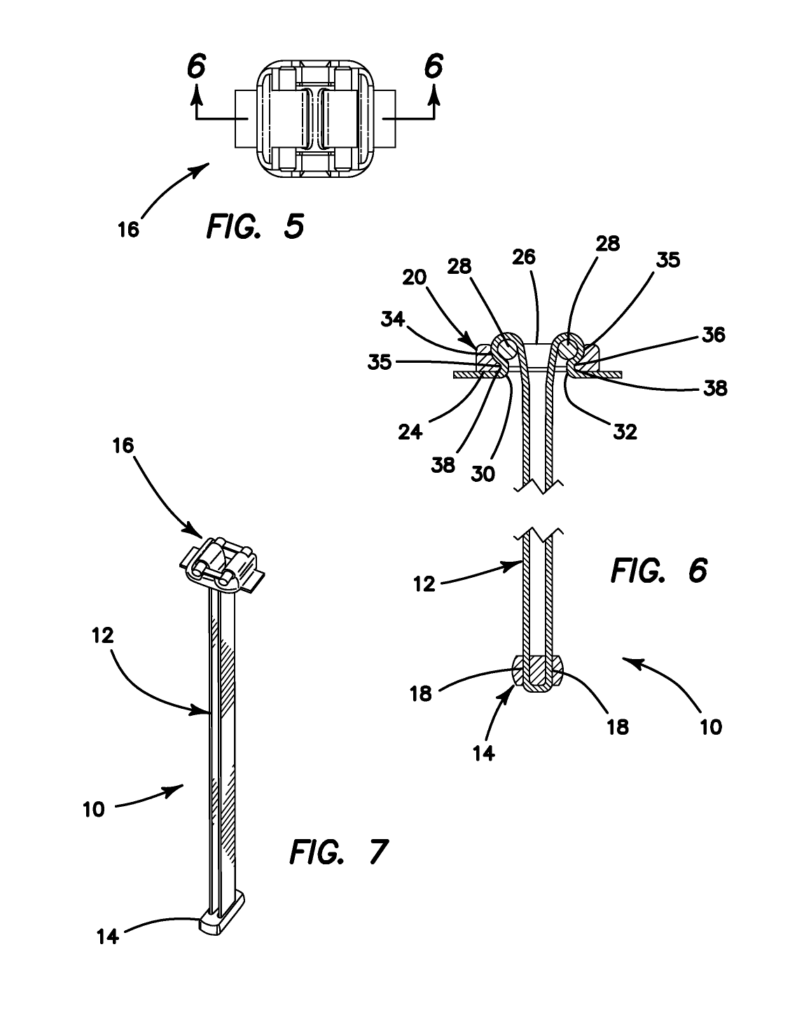

FIGS. 5-7 illustrate the apparatus 10 of FIGS. 1-4 through different views of the apparatus 10 with the lock 16 in a closed orientation. As illustrated, FIG. 6 is a cross-sectional view taken along lines 6-6 of FIG. 5.

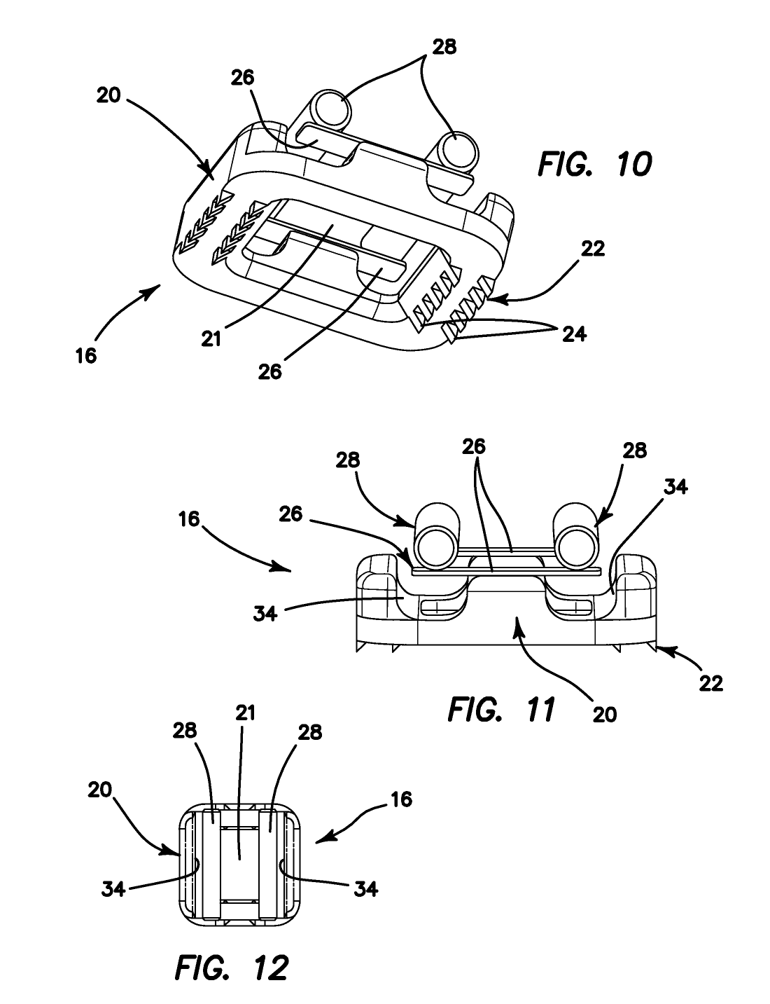

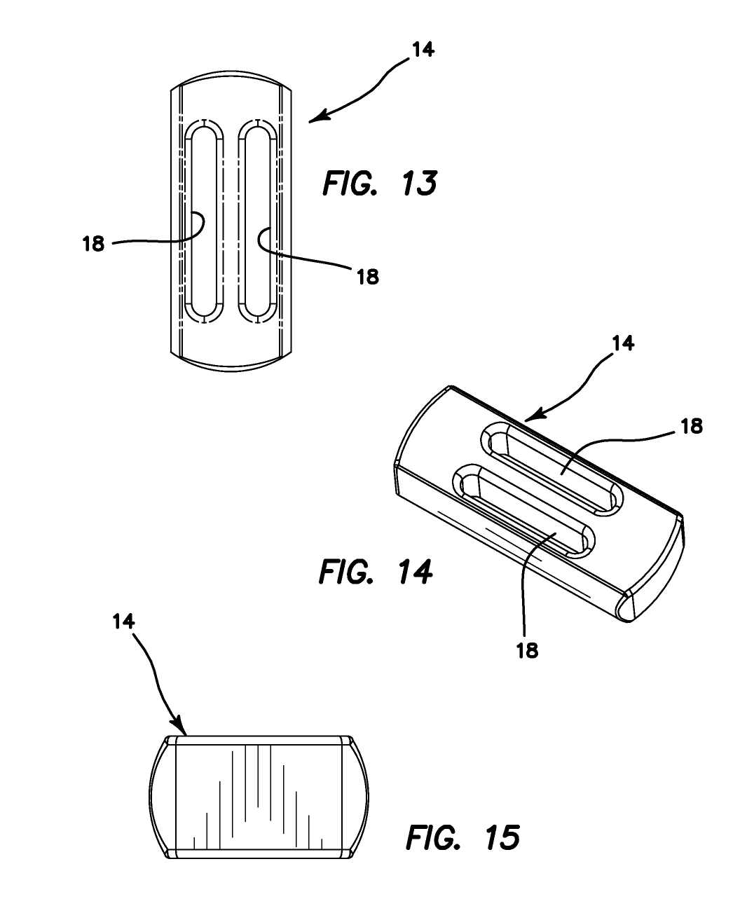

FIGS. 8-12 show various views of the lock 16 of the present invention, while FIGS. 13-15 show several views of the narrow button 14. As shown particularly in FIGS. 8-12, the lock 16 comprises a frame 20, a central or intake opening 21, a suture comb 22 having teeth 24, a plate 26, and two movable members or bars 28 disposed on the plate 26. As noted above, the lock 16 is constructed, in one embodiment, of 316 LVM stainless steel. The plate 26, with the two bars 28 disposed thereon, is attached to the frame 20 with four rows of teeth 24 comprising the comb 22.

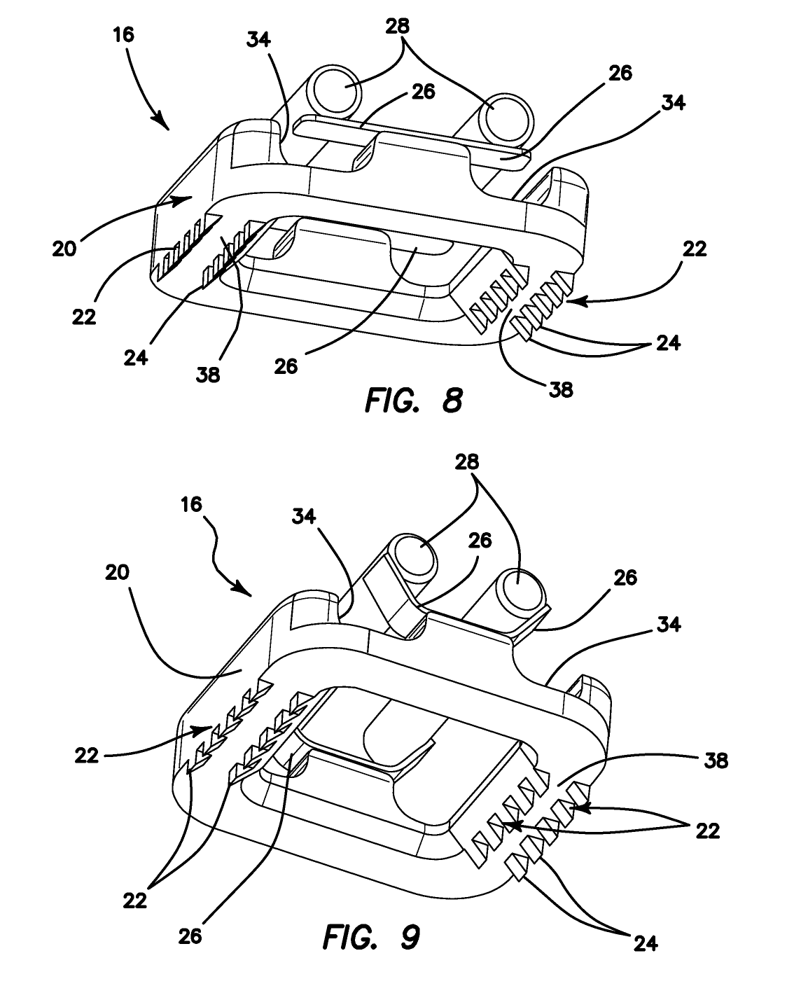

During use, when the two free ends 30, 32 of the suture 12 (see FIGS. 2-4) are tensioned, the bars 28 are moved into their locking position and the integrated suture comb feature 22 holds the suture in place. FIG. 3 shows the bars 28 in their open position, while in FIG. 6, the bars have moved into their locking position, wherein each bar 28 has been pulled by the tension placed on the suture ends 30, 32 toward respective slots 34 (FIG. 3). FIGS. 8, 10, and 11 show the lock 16 with the bars 28 in their locked position, while FIG. 9 shows the lock 16 with the bars 28 in their open position. As can be seen by comparing FIGS. 8 and 9, for example, the plates 26 are initially disposed in an upward, generally U-shaped configuration when the bars 28 are in their open position. The band 12 extends through the intake opening 21 of the lock frame 20, around the respective bars 28, as shown, then through the respective outer or exit slots 34. When a predetermined level of tension is applied to the free suture ends 30, 32, that applied tension moves the plates 26 to a substantially horizontal position, as shown in FIG. 8, thus moving the bars 28 mounted thereon downwardly toward the respective slots 34.

With further consideration of FIG. 6, the locking mechanism can be better understood. As noted above, tension is applied to the suture tails 30 and 32, which extend around respective bars 28, as shown, causing the plates 26 to move downwardly to a horizontal orientation, thereby moving the bars 28 downwardly toward slots 34. This downward movement causes the gap 34 between each bar 28 and the lock frame 20 to be diminished to an extent that the suture extending through the gap becomes clamped between the bar 28 and the frame 20, which may be identified as the first lock point, as shown at reference numeral 35. So, the first lock point essentially coincides with the opening of the slot 34. This is because the closure of the slots 34 by the downward movement of the bars 28 upon application of a predetermined tension level on the free suture ends 30, 32 creates the first lock point 35 by forcing the suture ends 30, 32, respectively against the inner frame surface forming the slot 34. A second lock point 36 occurs as the suture wraps around the bar 28 and passes around a corner formed on the inner surface of the frame, as shown. The wrapping of the suture around the inside surface of the lock frame creates this second lock point 36. A third lock point 38 is created as the suture passes over the teeth 24 and the teeth become embedded in the suture.

In one particular embodiment, the frame of the lock assembly 16 is generally square, with outside dimensions of 0.315 inches X 0.315 inches. Of course, these dimensions are not required, and may be varied as desired for particular applications.

Now with reference particularly to FIGS. 13-15, the narrow button 14 is shown in greater detail. In one particular embodiment, the frame of the narrow button may have dimensions of 0.313 inches in length and 0.128 inches in width., though, as noted above, these dimensions are not required and may be varied as desired for particular applications. Like the lock 16, the narrow button 14 may also be constructed of 0.316 LVM stainless steel, though again, different materials may be used. The slots 18 are adapted to receive the suture band 12 threaded therethrough, as is shown, for example, in FIGS. 1, 3, 4, 6, and 7. In preferred approaches, the narrow button 14 is supplied pre-threaded with the suture band 12 prior to use.

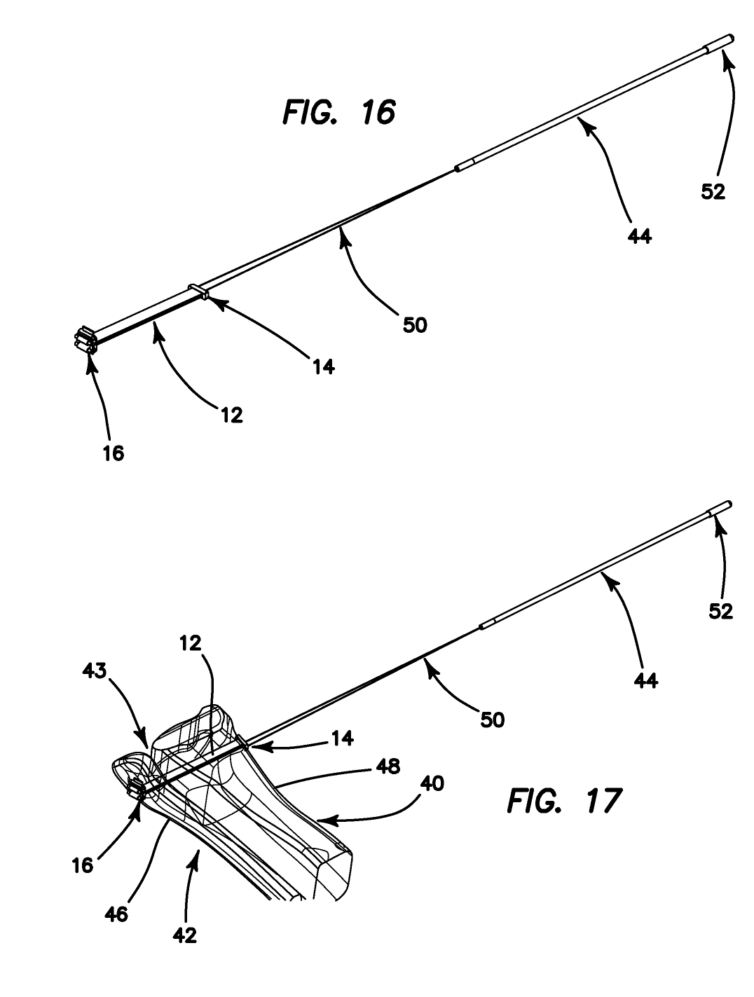

Referring now to FIGS. 16 and 17, in addition to the foregoing FIGS. 1-15, methods for using the apparatus 10 and associated instrumentation will now be described. The implant 10 is a sterile device using, as noted above, common implant materials, such as stainless steel for titanium for the lock 16 and narrow button 14, and a flat band 12 constructed of polyester suture material. A hole is pre-drilled through the cortices of the tibia 40 and fibula 42 (FIG. 17) from the open lateral side 46, for the purpose of repairing a separated syndesmosis 43. After complete reduction of the tibial and fibula bones, a straight guide needle 44 is passed through the drilled hole in the tibia and fibula and through the skin on the medial side 48, taking care to register the narrow button 14 into the pre-drilled hole. The lead narrow button 14 is pulled horizontally through the drill hole by applied tension onto a length of pull-through suture 50, until exiting the distal cortical surface, so that it slips across and engages onto the medial tibial cortex 48. At this point, the pull-through suture is cut, together with the associated needle 44, and discarded. Pulling the suture band 12 lightly on each end brings the lateral lock 16 into position resting flatly on the fibula. A final pull with sufficient force will fully engage the lock. Then, the suture ends are cut to complete the procedure.

After passing the narrow button 14 through the pre-drilled holes by means of the pass through needle 44, reduction of the ankle joint is achieved by applying tension to the band and the metallic button. Fixation forces are activated by pulling on the suture tails after removing the pass-through needle and pass-through suture, as discussed above. A hand-held stainless steel tensioning instrument may be utilized if higher tension levels are necessary to meet physiologic requirements.

In one particular embodiment, the straight needle 44 is made from 302 stainless steel that is used in most surgical needles. The needle may be 6 inches in length, and have a diameter of 0.0775 inches. A 5 inch (PET) suture loop 50 may be used to attach the needle and the narrow button 14, as shown. After placement of the narrow button 14, the suture loop 50 and straight needle 44 are discarded. A plastic protective cap 52 may be provided to protect the sharp end of the needle 44 before use. This cap 52 is removed at the outset of the procedure.

Though the foregoing procedure is described in connection with a Syndesmosis procedure for the repair of a fractured tibia and fibula, it is applicable, of course, to other areas of the body requiring repairs of a similar nature.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention, which is to be limited only in accordance with the following claims.

What is claimed is:

1. An implant system for stabilized fixation of tissue, comprising:
a button having a slot;
a band threaded through said slot; and
a lock comprising a frame having an intake opening, a movable member, and an exit slot, wherein a free end of material forming the band enters the lock through the intake opening, extends around the movable member, and then through the exit slot;
wherein the movable member is movable between an open position and a locking position.

2. The implant system as recited in claim 1, wherein the button comprises a pair of slots and the band is threaded through each of the pair of slots in sequence, so that the band is wrapped around a portion of the button separating the two slots.

3. The implant system as recited in claim 1, wherein the band is formed of suture material.

4. The implant system as recited in claim 2, wherein the band comprises a length of flat suture material threaded through the pair of slots of the button and through the intake opening and slots of the lock so that between the button and the lock the band comprises the length of suture doubled over itself.

5. The implant system as recited in claim 1, wherein the lock further comprises a plate on which the movable member is disposed, the plate having a first configuration when the movable member is in its open position and a second configuration when the movable member is in its locking position, the plate being movable from its first configuration to its second configuration responsive to applied tension exceeding a predetermined value.

6. The implant system as recited in claim 3, wherein the movable member is adapted to move toward the exit slot when it is moved from its open position to its locking position to narrow an effective width of the slot and to thereby clamp the free end of the suture band extending through the slot between the movable member and an inside surface of the outer portion of the frame to lock the suture band free end in place.

7. The implant system as recited in claim 6, wherein the lock is designed to clamp the free end of the suture band in place at multiple spaced lock points.

8. The implant system as recited in claim 7, wherein the first lock point is within the exit slot at a point where the movable member and the inside surface of the outer portion of the frame first clamp the suture band in place, and a second lock point, spaced distally from the first lock point, is created by a corner formed on the inside surface of the outer portion of the frame, so that the suture band is engaged between the corner and the movable member.

9. The implant system as recited in claim 8, and further comprising a third lock point, spaced distally from the second lock point, and created by engagement of the suture band with a row of teeth, comprising a suture comb, disposed on a surface of the lock frame.

10. The implant system as recited in claim 9, wherein the frame comprises a second movable member and a second exit slot, the two exit slots being disposed on opposite sides of the intake opening adjacent to respective opposed outer portions of the frame.

11. The implant system as recited in claim 10, wherein the lock comprises a second suture comb, each suture comb being disposed on the frame of the lock adjacent to respective exit points for the free ends of the suture bands from each of the two exit slots.

12. The implant system as recited in claim 6, wherein the movable members comprise bars.

13. The implant system as recited in claim 2, and further comprising a suture comb disposed on a surface of the frame.

14. The implant system as recited in claim 13, wherein the suture comb comprises a row of teeth for engaging suture passing thereover, and clamping the suture in place.

15. The implant system as recited in claim 14, wherein the suture comb comprises multiple rows of said teeth.

16. A lock for use in an implant system for stabilized fixation of tissue, the lock comprising:
a frame having an intake opening, a movable member, and an exit slot, for creating a path for a band of material to extend along, the path extending from the intake opening, around the movable member, and exiting through the exit slot, the exit slot being defined in part by an inner surface of the frame;
wherein the movable member is movable between an open position and a locking position.

17. The implant system as recited in claim 16, wherein the lock further comprises a plate on which the movable member is disposed, the plate having a first configuration when the movable member is in its open position and a second configuration when the movable member is in its locking position, the plate being movable from its first configuration to its second configuration responsive to applied tension exceeding a predetermined value.

18. The implant system as recited in claim 17, wherein the movable member is adapted to move toward the exit slot when it is moved from its open position to its locking position to narrow an effective width of the slot and to thereby clamp the free end of a suture band extending through that slot between the movable member and an inside surface of the outer portion of the frame to lock the suture band free end in place.

19. The implant system as recited in claim 18, wherein the lock is designed to clamp the free end of the suture band in place at multiple spaced lock points.

20. The implant system as recited in claim 19, wherein the first lock point is within the exit slot at a point where the movable member and the inside surface of the outer portion of the frame are first adapted to clamp the suture band in place, and a second lock point, spaced distally from the first lock point, is created by a corner formed on the inside surface of the outer portion of the frame, so that the suture band is adapted to be engaged between the corner and the movable member.

21. The implant system as recited in claim 20, and further comprising a third lock point, spaced distally from the second lock point, and created by engagement of a suture band, extending along the pathway through the lock, with a row of teeth, comprising a suture comb, disposed on a surface of the lock frame.

22. The implant system as recited in claim 21, wherein the frame comprises a second movable member and a second exit slot, the two exit slots being disposed on opposite sides of the intake opening adjacent to respective opposed outer portions of the frame.

23. The implant system as recited in claim 22, wherein the lock comprises a second suture comb, each suture comb being disposed on the frame of the lock adjacent to respective exit points for the free ends of the suture bands from each of the two exit slots.

24. The implant system as recited in claim 16, and further comprising a suture comb disposed on a surface of the frame.

25. The implant system as recited in claim 24, wherein the suture comb comprises a row of teeth for engaging suture passing thereover, and clamping the suture in place.

26. The implant system as recited in claim 25, wherein the suture comb comprises multiple rows of said teeth.

27. A lock for use in an implant system for stabilized fixation of tissue, the lock comprising:
a frame having an opening for receiving a length of material therethrough, a movable member about which the length of material may extend, and a slot through which the length of material may exit after passing about the movable member, the slot being defined in part by an inner surface of the frame;
wherein the movable member is movable between an open position and a locking position, the open position being adapted to permit free movement of the length of material through the lock and the locking position being adapted to clamp the length of material in place;
the lock being constructed and designed to clamp the length of material at a plurality of locking points, spaced from one another, when the movable member is in said locking position.

28. A method for effecting repair of tissue using a tissue fixation band apparatus comprising a lock, a button, and a suture band, under tension, extending between the lock and the button, comprising:
pulling the button through a hole extending through the tissue to be repaired, until it exits a distal surface of the tissue and engages the distal surface of the tissue;
pulling the suture band on each end to locate the lock in position on a proximal surface of the tissue;
applying tension to free ends of the suture band exiting from the lock of the fixation band apparatus to tension the suture band to a desired level; and
causing a movable clamping member within the lock to move to a locking position upon application of the desired tension level to the suture band, to thereby engage the lock and clamp the suture band in place at the desired tension level.

29. The method as recited in claim 28, wherein the causing step creates a plurality of spaced locking points along a length of the suture band free end extending through the lock.
